(19) United States
(12) Patent Application Publication
(10) Pub. No.: US 2019/0110933 A1
(43) Pub. Date: Apr. 18, 2019

(54) SYSTEM AND METHOD FOR TREATING SURFACES OF BODIES, IN PARTICULAR FOR WOUND TREATMENT

(71) Applicant: LEIBNIZ-INSTITUT FUR PLASMAFORSCHUNG UND TECHNOLOGIE E.V., Greifswald (DE)

(72) Inventors: Klaus-Dieter WELTMANN, Binz (DE); Thomas VON WOEDTKE, Sundhagen (DE); Manfred STIEBER, Greifswald (DE); Stefan HORN, Loissen (DE); Philipp TURSKI, Greifswald (DE); Ronny BRANDENBURG, Grosse Kiesow OT Kessin (DE)

(73) Assignee: LEIBNIZ-INSTITUT FUR PLASMAFORSCHUNG UND TECHNOLOGIE E.V., Greifswald (DE)

(21) Appl. No.: 16/090,228

(22) PCT Filed: Mar. 28, 2017
(86) PCT No.: PCT/EP2017/057310

§ 371 Date: Sep. 29, 2018

(30) Foreign Application Priority Data
Mar. 30, 2016 (DE) 10 2016 105 759.2

(51) Int. Cl. A61F 13/02; A61N 1/44; A61F 13/00; A61M 1/00

(52) U.S. Cl. CPC A61F 13/0216; A61N 1/44; A61B 18/042; A61M 1/0088; A61M 1/0025; A61F 13/00068

(57) ABSTRACT

The invention relates to a system for treating surfaces of bodies, in particular a vacuum dressing, comprising at least one connection means (5a) for the fluidic attachment of a negative-pressure generator for generating negative pressure in a space that can be positioned on the surface of a body, and at least one means for plasma generation (8, 8a, 15) by which the space in which negative pressure can be generated can be at least partly filled with plasma or a plasma-activated medium.

The device according to the invention is particularly suitable for the combined negative pressure and plasma treatment of a wound.

Furthermore, the invention relates to a method for treating surfaces of bodies by means of the system according to the invention, wherein a body with a surface to be treated is provided, the system for treating surfaces of bodies is provided, a negative pressure is generated in a space positioned on the surface of the body and the space is filled at least partly with plasma or plasma-activated medium.

The invention relates to a system for treating surfaces of bodies, in particular for wound treatment by means of a combined plasma and negative pressure treatment, and a corresponding method for treating surfaces of bodies. The system consists in particular of a vacuum dressing for combined plasma and negative pressure treatment.

The treatment of complicated or chronic wounds by means of negative pressure wound therapy (also referred to as “vacuum therapy”, “negative pressure wound therapy, NPWT” or “Vacuum Assisted Closure, V.A.C.”) was first described in 1991 in the patents of the Wake Forest University Winston-Salem by Louis C. Argenta and Michael J. Morykwas (e.g. WO 9309727 A1 and WO 9420041 A1). This was followed by numerous further patents and publications by the latter and other authors, which describe developments of this method and its applications. In this application the wound is exposed to negative pressure, whereby pathological wound secretions are drawn off and the wound healing is stimulated or supported. By means of suitably implemented devices the following effects are mainly achieved, which contribute to the improved healing of chronic problematic wounds:
reduction of the accumulation of fluid in the tissue space (wound edema),
improvement of oxygen saturation in the tissue,
improvement of local blood circulation,
reduction of bacterial growth (colonization),
reduction in amount of cellular decomposition products (cell detritus),
stimulation of cell growth by means of mechanical stress,
promotion of the formation of granulation tissue.

Negative pressure wound therapy is described in particular in the following publications:
Argenta L C, Morykwas M J (1997) Vacuum-assisted closure: a new method for wound control and treatment: clinical experience. Ann Plast Surg 38:563-76
Morykwas M J, Argenta L C, Shelton-Brown El (1997) Vacuum-assisted closure: a new method for wound control and treatment: animal studies and basic foundation. Ann Plast Surg 38:553-62
Horch, R. and M. Leffler, Grundlagen, Indikationen, grundlegende therapeutische Konzepte and Kontraindikationen bei der Wundbehandlung mit der Vakuumtherapie, in Manual der Wundheilung: Chirurgisch-dermatologischer Leitfaden der modernen Wundbehandlung, T. Wild, Auböck, Josef (Hrsg.) Editor. 2007. p. 123-128 [Basic principles, indications, basic therapeutic concepts and contraindications for wound treatment by vacuum therapy, in the Manual for wound healing: surgical-dermatological guidelines for modern wound treatment].
Horch, R. E., et al., Lokale Unterdrucktherapie im Wundmanagement, [Local negative pressure therapy in wound management] in EWMA position document 2007, European Wound Management Association (EWMA). London: MEP Ltd,. p. 1-17.
Probst, W. and A. Vasel-Biergans, 10. Besondere Verfahren der Wundbehandlung; 10.1 Lokale Unterdrucktherapie, in Wundversorgung für die Pflege—Ein Praxisbuch; 2011, Wissenschaftliche Verlagsgesellschaft p. 319-326. [Special methods for wound treatment: Local negative pressure therapy in wound management for care—A practice book]
Protz, K. Tiefe Wunden and Wundhöhlen, [Deep wounds and wound cavities] Der Hausarzt 08/2014, S.52-54.

The successes described in the various studies and case reports which relate to the treatment of wounds that are difficult to heal and evidence of the effectiveness of negative pressure therapy compared to conventional methods of wound treatment for severe wound problems, mainly by reducing the periods required for wound healing and care, have resulted in said methods becoming established in clinical practice on the basis of commercially available systems.

A negative pressure therapy system of this kind generally consists of a vacuum dressing and a control device with a vacuum pump for controlling the generation of negative pressure. The vacuum dressing is a combination of a foam wound dressing (made of polyurethane or polyvinyl), a transparent film for sealing and covering the vacuum dressing and a suction drain connected to said film. By means of the vacuum system it is possible to generate defined negative pressure continually or in a pulsed manner. The system is completed by a collection vessel for receiving the fluid suctioned from the wound.

The solutions known from the prior art have the disadvantage in particular that although they do achieve reduced bacterial growth, without an additional instillation treatment (as described in patent documents DE 19 722 075 C1 and DE 10 2013 226 708 A1) there is no significant reduction of bacteria in the wound area.

A further disadvantage of the latter is that to assess the state of the wound or the success of the treatment the vacuum dressing has to be opened, which on the one hand disturbs the wound healing process and on the other hand risks further infection. In addition, for many wounds (e.g. burns) changing the dressings causes the patient a lot of pain.

This has led to the problem of providing a system and a corresponding method for treating surfaces of bodies which represents an improvement over the described disadvantages of the prior art.

Said problem is addressed by the system according to claim 1 and the method according to claim 14. Advantageous embodiments of the system are the subject matter of subclaims 2 to 13 and an advantageous embodiment of the method is claimed in subclaim 15. The invention is described in the following.

A first aspect of the invention relates to a system for treating surfaces of bodies, in particular for wound treatment, comprising at least one connection means for connecting fluidically a negative pressure generator for generating negative pressure in a space which can be positioned on the surface of a body and at least one device for plasma generation, by means of which the space in which negative pressure can be generated can be filled at least partly with plasma or a plasma-activated medium.

The space can be defined by a suitable chamber provided by the system or by a porous material, such as e.g. foam, in which negative pressure is formed. In an additional embodiment the system for treating surfaces of bodies also comprises the negative pressure generator.

The system according to the invention is in particular a vacuum dressing for treating a wound of a human or animal body surface.

In particular according to the invention a device for negative pressure wound therapy is combined with an antimicrobial and wound-healing atmospheric pressure plasma source, and optionally with a device for sensor-based wound monitoring to form a new system.

For this e.g. a device for negative pressure wound therapy can be combined with a surface plasma source for generating a dielectric barrier discharge (DBD), which together with foam-wound dressings of the device for negative pressure wound therapy is positioned in a sandwich arrangement in the wound area. The plasma source used for this for generating a dielectric barrier discharge is preferably made from flexible materials, such that it can cling with the wound dressings to the surface of the wound.

The system according to the invention enables essentially a more efficient treatment of complicated or chronic wounds compared to the use of a conventional negative pressure wound therapy device. Here the application of the system according to the invention and the method performed thereby is not limited to medical purposes, but can be used in any situation in which effective and efficient microbial reduction is required.

In particular, the device for the negative pressure generation of the system according to the invention can be designed in a conventional manner and reference is thus made to the relevant publications, the content of which relating to negative pressure is thus included in the present patent application.

In addition to reducing bacterial growth by means of conventional negative pressure therapy, with the additionally used plasma source on the one hand there is a significant reduction of the bacteria in the wound area and on the other hand the healing of the wound is promoted.

In particular, by combining a device for negative pressure therapy with a device for generating a plasma discharge it is possible to effectively avoid MRSA infections.

A further advantage is that by using the integrated sensor system to assess the state of the wound or the success of the treatment the vacuum dressing does not need to be opened prematurely and thus on the one hand the wound healing process can remain uninterrupted and on the other hand the risk of further infection is reduced.

The connection means of the system according to the invention for treating surfaces of bodies can be connected fluidically for example via a hose line, e.g. a drainage hose, to a negative pressure generator, such as e.g. a vacuum pump.

According to one embodiment the system for treating surfaces of bodies comprises a first device for plasma generation, which is arranged in the space in which negative pressure can be generated so that the plasma and/or plasma-activated medium can be produced in said space.

In this embodiment thus the plasma or plasma-activated medium can be produced directly on the surface to be treated.

According to a further embodiment the first device for plasma generation is configured as a sensor system for detecting at least one physical parameter on the surface of the body. This means that the physical parameter can be detected in particular on the surface or in the projection area of the surface, but also in the space positioned on the surface of the body. By means of the physical parameter detected in this way it is possible in particular to ascertain the state of a wound of the body on which the system for treating the surfaces of bodies is arranged, e.g. as part of a wound monitoring process. Suitable physical parameters for this include in particular the temperature, pressure, humidity and/or pH value.

This means that the first device for plasma generation has a double function, namely the generation of plasma or plasma-activated medium on the surface to be treated and the determination of at least one physical parameter on the surface of the body, wherein only one of said functions is performed per unit of time.

The basis for measuring parameters to assess the state of the wound, such as temperature, humidity, pressure and pH value, is the detection and processing of changes in the electrical DC or AC levels (ohmic resistance, impedance, capacitance) in the electroconductive components, the outer electrode and/or inner electrode.

In particular, the system according to the invention for treating surfaces of bodies is configured to switch from an active plasma generation mode into a sensor mode, e.g. by means of a microprocessor-based process controller.

Furthermore, the system for treating surfaces of bodies can comprise a second device for plasma generation, which is arranged externally in relation to the space in which negative pressure can be produced and is connected by means of a fluidic connection to the space so that the plasma or the plasma-activated medium or plasma-activated medium can be generated outside the space and can be directed into the space. This means in particular that the plasma can be generated outside of the space and the plasma-activated medium formed by means of the plasma can be directed into the space.

In this embodiment thus the plasma or plasma-activated medium can be produced remotely from the surface to be treated and supplied to the surface. In this way thus a device for negative pressure wound therapy is combined with an external plasma source connected e.g. via a hose line to the system according to the invention for treating surfaces, in particular the vacuum dressing. The second device for plasma generation, i.e. the external plasma source, can be configured to generate a dielectric barrier discharge. Alternatively to this the second device for plasma generation can also be e.g. a jet plasma source operated in particular in the kHz, MHz or GHz range.

In particular, the system can be configured in the described embodiment to suction or supply a plasma-activated medium generated by the plasma source (e.g. gas or aerosol) at the same time or alternately by means of a preferably microprocessor-based process controller.

The first device for plasma generation, i.e. the internal plasma source and the second device for plasma generation, i.e. the external plasma source, are used in particular alternatively to one another for plasma generation. However, they can also be used in combination with one another.

According to a further embodiment the system for treating surfaces of bodies comprises a porous material, in particular foam, which defines the space in which negative pressure can be generated. Of course, the porous material can contract with the application of negative pressure, so that the space defined by the porous material can fluctuate according to the presence of negative pressure.

In a further embodiment the system for treating surfaces of bodies comprises a chamber which defines the space in which negative pressure can be generated. In this case the term chamber is used in particular to define a cavity of the system, which together with the surface of the corresponding body to be treated forms the said space.

The system for treating surfaces of bodies can also comprise a negative pressure generator, in particular a vacuum pump, for generating negative pressure in the space, wherein the negative pressure generator can be connected or is connected fluidically to the space by means of the connection means.

According to a further embodiment the device for plasma generation, in particular the first or the second device for plasma generation, is designed to generate a dielectric barrier discharge.

In a further embodiment the device for plasma generation is made of flexible materials which are designed to lie on the surface of the body, wherein the flexible materials form at least part of the space.

Furthermore, according to a further embodiment the system for treating surfaces of bodies comprises a first layer made of flexible materials, which is designed to be applied to the surface of the body, and the system comprises a second layer made of flexible materials, wherein the device for plasma generation is designed to be flat and is arranged between the first layer and the second layer, and wherein the first layer, the device for plasma generation and the second layer form the space in which negative pressure can be produced.

The device for generating plasma can comprise at least one inner electrode and at least one earthed outer electrode.

In this case the outer electrode can comprise an electroconductive textile, in particular a woven material, or can be made from the latter.

According to a further embodiment the outer electrode can comprise electroconductive gauze or consists of the latter.

According to a further embodiment of the invention the device for plasma generation comprises a plurality of spacer elements made from an electrically insulating material, wherein the spacer elements define a distance between the inner electrode and the outer electrode.

The described embodiments of the device for plasma generation are particularly suitable for use as a flat flexible plasma source and thus for use as an internal plasma source in the system according to the invention for treating surfaces of bodies.

Furthermore, according to one embodiment the system for treating surfaces of bodies comprises a film, in particular a transparent film, for sealing and covering the system from the environment, wherein in particular the film has at least one opening to which the connection means is connected so that the negative pressure generator is fluidically connected through the opening to the space in which negative pressure can be generated.

A second aspect of the invention relates to a method for treating surfaces of bodies, in particular for treating surfaces of human or animal tissue outside the human or animal body, wherein a body with a surface to be treated is provided, a system for treating surfaces of bodies according to the first aspect of the invention is provided, negative pressure is generated in a space positioned on the surface of the body, in particular by means of a negative pressure generator of the system for treating surfaces connected to the connection means and the space is filled at least partly with plasma or plasma-activated medium, wherein the plasma or the plasma-activated medium is generated in particular by means of the device for plasma generation of the system for treating surfaces.

This means that in particular according to this aspect of the invention the method is not performed in or on the human body but outside the human or animal body.

According to one embodiment of the method the space is filled alternately to negative pressure generation with the plasma or the plasma-activated medium. In this case the plasma or the plasma-activated medium can preferably be positioned in the space periodically alternately to the negative pressure generation. In addition, by means of the first device for plasma generation the function of a sensor can be performed, in particular if said first device for plasma generation is not used for generating plasma.

When using the electrodes of the plasma source for sensor purposes in the wound area, e.g. for controlling the temperature, pressure, humidity or pH value, it is possible for example to switch from an active plasma generation mode to a sensor mode. For this a microprocessor-based process controller can be used for example.

In the method the plasma or the plasma-activated medium can be generated directly in the space positioned on the surface of the body and/or can be produced externally and directed to the said space.

A third aspect of the invention relates to the use of a system for treating surfaces of bodies according to the first aspect of the invention for treating surfaces of bodies, in particular human or animal bodies. The system consists in particular of a vacuum dressing. In particular, the surface to be treated is a wound on the human or animal body.

A fourth aspect of the invention relates to a method for treating surfaces of human or animal tissue in or on a human or animal body, wherein a body of human or animal tissue with a surface to be treated is provided, a system for treating surfaces of bodies is provided according to the first aspect of the invention, a negative pressure is generated in a space positioned on the surface of the body and the space is filled at least partly with plasma or plasma-activated medium. The system consists in particular of a vacuum dressing. In particular, the surface to be treated is a wound on a human or animal body.

According to one embodiment of the method the space is periodically filled alternately to negative pressure generation with the plasma or the plasma-activated medium. In this way the plasma or the plasma-activated medium can preferably be positioned periodically in the space alternately to the negative pressure generation. In addition, by means of the first device for generating plasma the function of a sensor can be performed, particularly if said first device for plasma generation is not used for generating plasma.

In the method the plasma or the plasma-activated medium can be generated directly in space positioned on the surface of the body and/or are generated externally and directed to the said space.

Further details and advantages of the present invention are described in the following description of embodiments. With the following embodiments shown in a series of drawings the concept of the invention and the schematic structure and the handling of the device system are explained in detail. The individual elements of the structure of the devices are labelled with the reference numerals listed below.

In the latter:

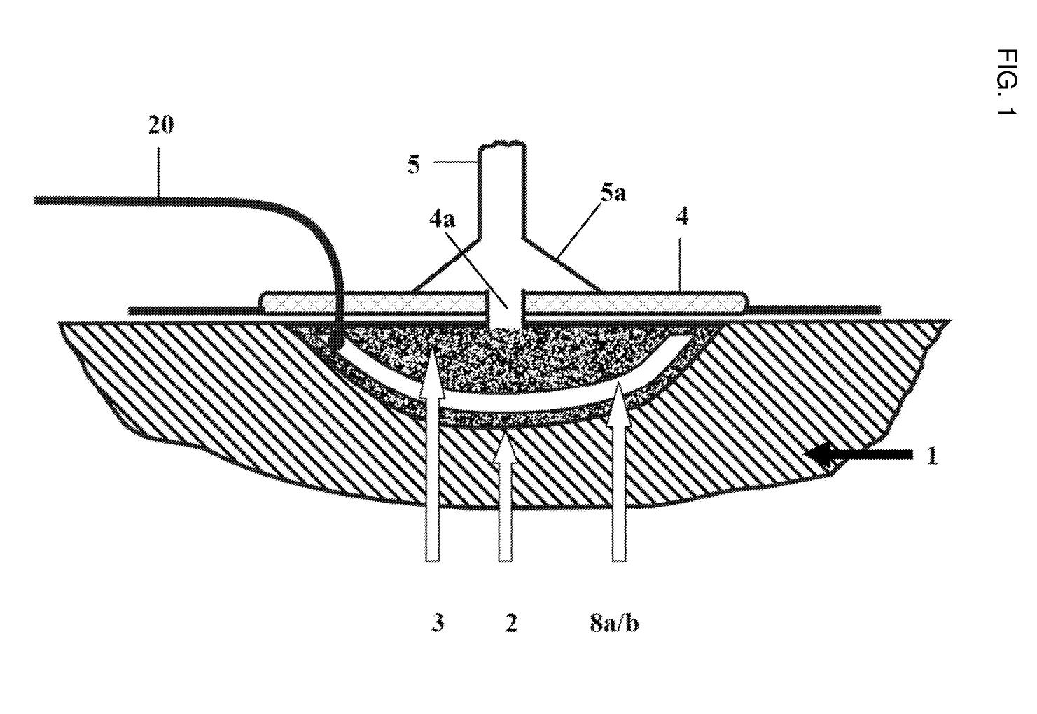

FIG. 1 is a cross-sectional representation of a system according to the invention for treating surfaces;

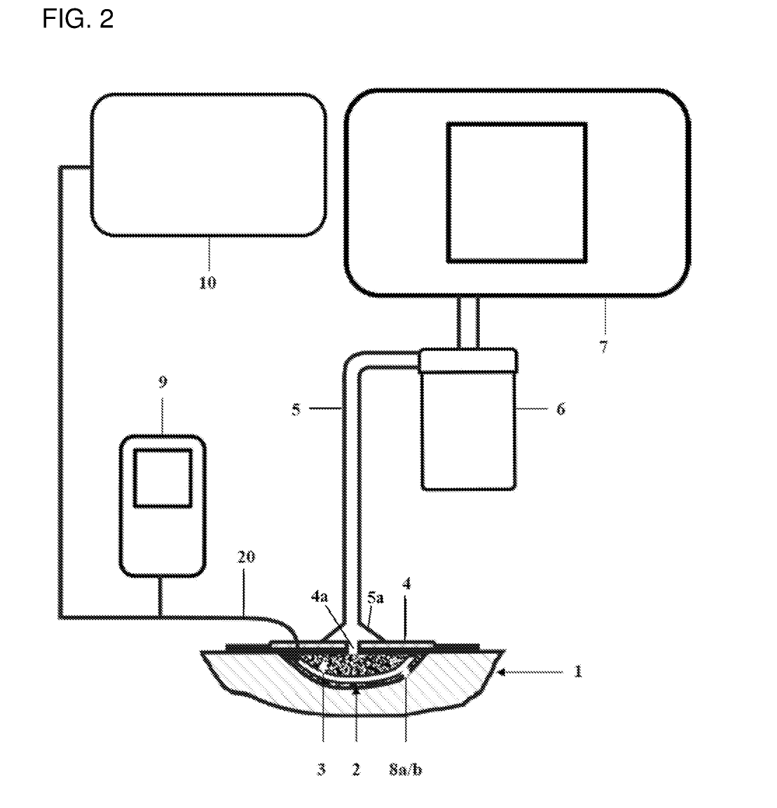

FIG. 2 is a schematic representation of a first embodiment of the system according to the invention with an internal plasma source;

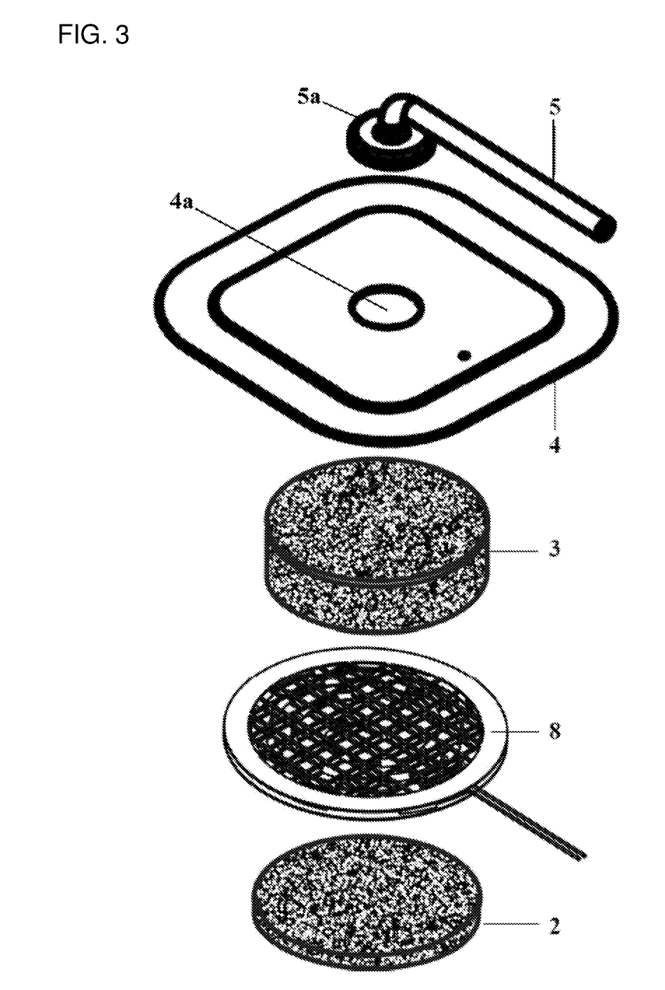

FIG. 3 is an exploded view of the first embodiment of the system according to the invention with an internal plasma source;

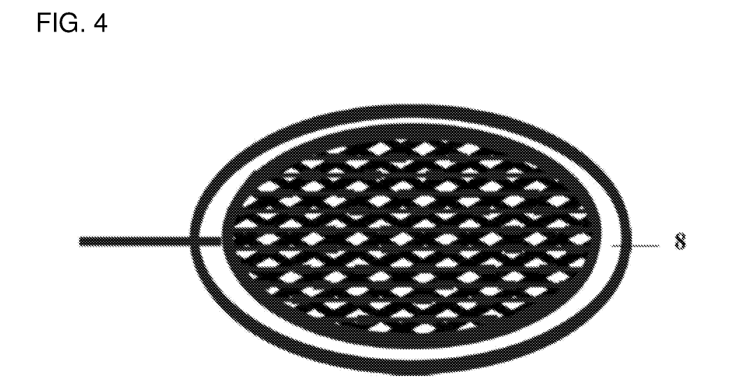

FIG. 4 is a perspective view of an embodiment of an internal plasma source according to the invention;

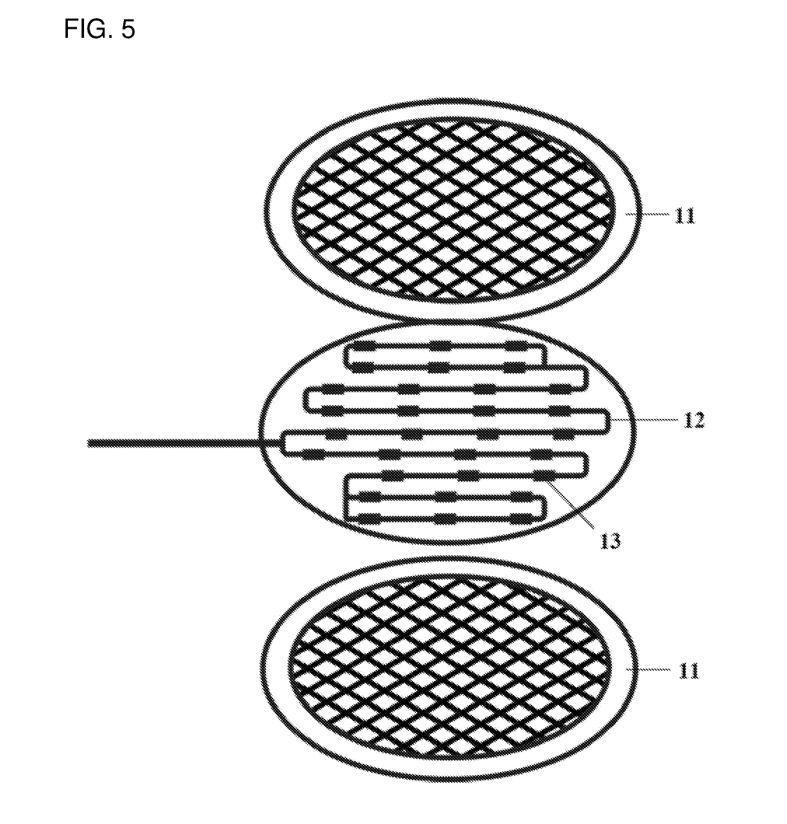

FIG. 5 is an exploded view of the embodiment shown in FIG. 4 of the internal plasma source;

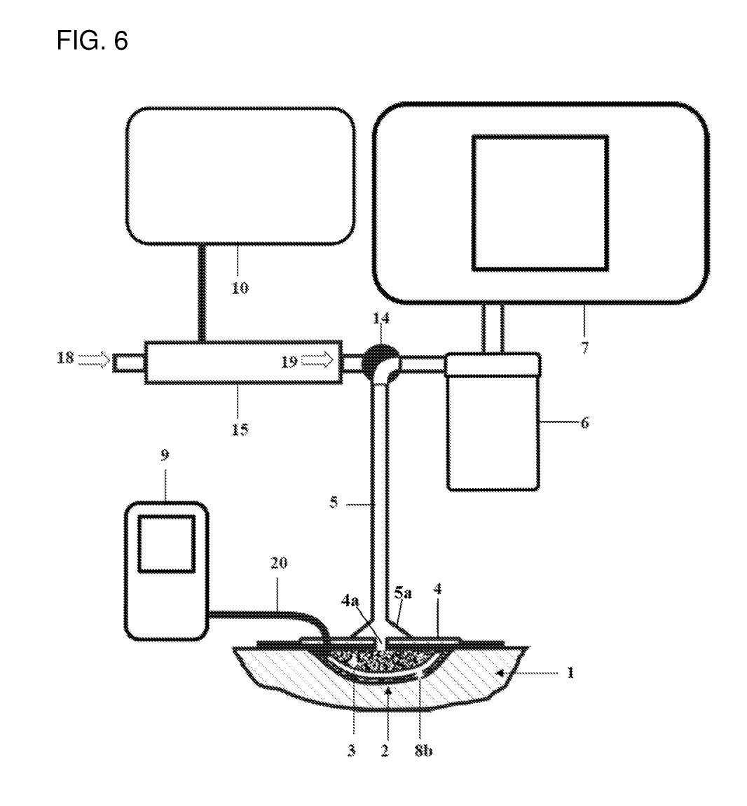

FIG. 6 is a schematic representation of a second embodiment of the system according to the invention with an external plasma source;

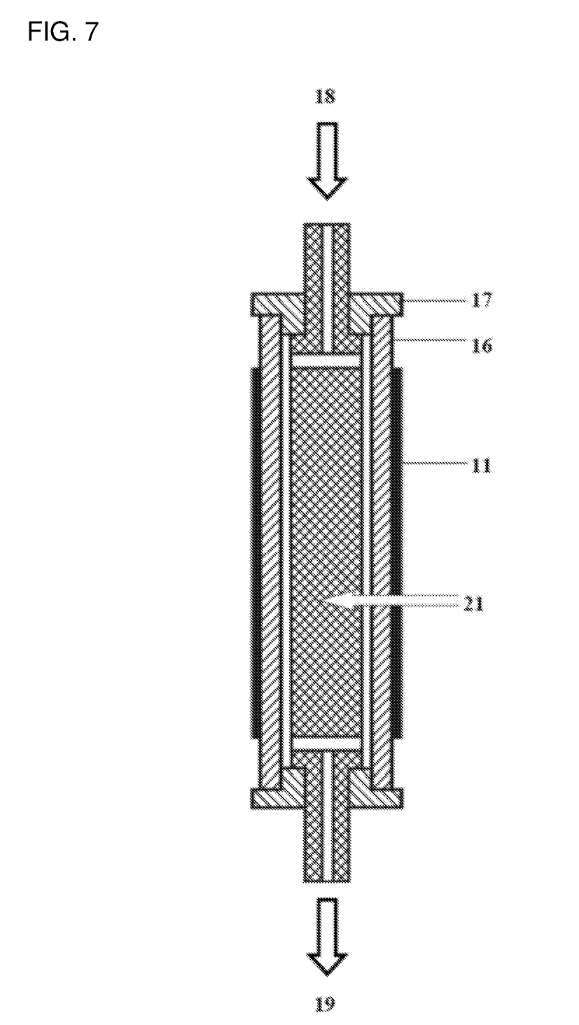

FIG. 7 is a cross-sectional representation of an external DBD-based plasma source.

FIG. 1 shows the usual basic arrangement of the essential components of a system according to the invention designed as a vacuum dressing on a wound of biological tissue 1 with a flexible, flat DBD-based plasma source 8a embedded between two foam wound dressings 2, 3, which plasma source can be used additionally as a sensor system 8b for wound monitoring. The wound is covered for example by a transparent film 4 and is sealed from the environment. Here the film 4 seals over the wound dressing 3 and the body surface surrounding the wound.

The film 4 comprises an opening 4a and an connection means 5a arranged over the opening 4a for the fluidic connection of a negative pressure generator for producing negative pressure in the space formed by the foam wound dressings 2, 3, e.g. via a drainage hose 5. At the same time the drainage hose 5 can be used for suctioning off wound fluid.

The plasma source 8a or the sensor system 8b is connected by an electric supply line 20 for connecting to a power supply 10 and/or a measuring and control device 9 for wound monitoring. By means of the shown plasma source 8a plasma can be generated directly in the space in which negative pressure is produced, i.e. in this case in the space formed by the foam wound dressings 2, 3.

FIG. 2 shows a diagram of the whole structure of the combined device system according to a first embodiment with an internal DBD-based plasma source 8a embedded in the vacuum dressing. The vacuum dressing is connected to a negative pressure-control device 7 with vacuum pump for the purpose of evacuating and/or suctioning off wound secretions in the usual way via an connection means 5a and via a drainage hose 5 and a collection vessel 6 for wound secretions, whereas the combination of a DBD-based plasma source 8a and a sensor system 8b is connected via corresponding electrical supply lines 20 on the one hand to a measurement and control device 9 for wound monitoring by means of the sensor system 8b and on the other hand to the power supply 10 for the plasma source 8a.

The exploded view shown in FIG. 3 of the first embodiment of the system according to the invention configured as a vacuum dressing for treating surfaces with an internal plasma source 8 is intended to illustrate the sandwich arrangement of the thin foam wound dressing 2, which lies directly on the wound, the combination 8 of a DBD-based plasma source and a sensor system, the overlying part of the foam-wound dressing 3 and the transparent film 4 for sealing and covering with the connection means 5a for the drainage hose 5 in the vacuum dressing.

FIGS. 4 and 5 show the basic structure of an embodiment of the combination 8 composed of different layers of a DBD-based plasma source and a sensor system in an assembled view (FIG. 4) and in an exploded view (FIG. 5).

A centrally meandering insulated electroconductor 12 is optionally used as part of a sensor system 8b or as a high voltage electrode covered by a dielectric, against which an earthed outer electrode 11 is applied on both sides at a defined distance, defined by the spacer elements 13 made of insulation material, which outer electrode is made from electroconductive gauze or electroconductive textile material, in particular woven material, and which can also be used optionally as part of the sensor system 8b for wound monitoring. Instead of the meandering arrangement of the electroconductor 12 encased in insulation material it is also possible to have a spiral-shaped arrangement or any arrangement in which plasma can be generated over the surface as far as possible in a plane with a corresponding electrode placement.

FIG. 6 shows a second embodiment of the system according to the invention for treating surfaces, i.e. a further way of combining a negative pressure therapy device with an external plasma source 15. Unlike the first embodiment shown in FIG. 2 here instead of the internal DBD-based plasma source 8a an external DBD-based plasma source 15 is used. In this case in the embodiment shown here the plasma-treated gas 19 generated by the plasma source 15 from the supplied gas 18 is supplied periodically in a defined amount to the vacuum dressing and thus the space in which negative pressure is generated via a three-way valve 14 through the drainage hose 5 and the connection means 5a.

After a defined reaction time via the three-way valve 14 the suction is performed with the negative pressure therapy device or the negative pressure generator, e.g. a vacuum pump as part of the negative pressure control device 7. In this case the electrode arrangement shown in FIG. 4 is also integrated into the vacuum dressing, but is not used here as a plasma source but only as a sensor system 8b in connection with the measuring and control device 9 for wound monitoring.

Many different embodiments of an external plasma source 15 can be used here.

FIG. 7 shows schematically by way of example the structure of a coaxial arrangement of a DBD-based plasma source in a cross-sectional view. In this case the high voltage carrying inner electrode 21 is a metal cylinder, which is surrounded by a glass tube 16 used as a dielectric and coated with an earthed outer electrode 11. The narrow gap between the glass tube 16 and the inner electrode 12, which is sealed externally by the centering and sealing rings 17, is used as a gas chamber in which the plasma is formed.

List of reference numerals

| | |
|---|---|
| 1 | biological tissue |
| 2 | foam wound dressing, part A |
| 3 | foam wound dressing, part B |
| 4 | film, in particular transparent film for sealing and covering |
| 4a | opening |
| 5 | drainage hose |
| 5a | connection means |
| 6 | collection vessel for wound secretion |
| 7 | negative pressure control device with vacuum pump |
| 8 | first device for plasma generation, in particular flexible, surface |
| | DBD-based plasma source or combination of plasma source and |
| | sensor system |
| 8a | first device for plasma generation, in particular flexible, surface |
| | DBD-based plasma source, |
| 8b | sensor system for wound monitoring |
| 9 | measurement and control device for wound monitoring |
| 10 | power supply for the plasma source |
| 11 | earthed outer electrode |
| 12 | high voltage conducting inner electrode coated with insulation |
| | material |
| 13 | spacer elements made of insulating material |
| 14 | three-way valve |
| 15 | second device for plasma generation, in particular external DBD- |
| | based plasma source |
| 16 | glass tube |
| 17 | centering and sealing ring |
| 18 | gas (e.g. air) |
| 19 | plasma-treated gas |
| 20 | electric supply lines |
| 21 | metal inner electrode |

1. A system for treating surfaces of bodies, in particular a vacuum dressing, comprising
at least one connection means (5a) for creating a fluidic connection of a negative pressure generator for generating negative pressure in a space which can be positioned on the surface of a body and
at least one device for plasma generation (8, 8a, 15), by means of which the space in which negative pressure can be generated can be filled at least partly with plasma or a plasma-activated medium.

2. The system for treating surfaces of bodies according to claim 1, characterized in that the system comprises a first device for plasma generation (8, 8a), which is arranged in the space in which negative pressure can be generated, so that the plasma and/or plasma-activated medium can be generated in the space.

3. The system for treating surfaces of bodies according to claim 1, characterized in that the first device for plasma generation (8, 8a) is configured as a sensor system (8b) for detecting at least one physical parameter on the surface of the body.

4. The system for treating surfaces of bodies according to claim 1, characterized in that the system comprises a second device for plasma generation (15), which is arranged externally in relation to the space in which negative pressure can be generated, and is connected by a fluidic connection to the space, so that the plasma-activated medium can be generated outside the space and can be directed into the space.

5. The system for treating surfaces of bodies according to claim 1, characterized in that the system comprises a porous material, in particular foam, which defines the space in which negative pressure can be generated.

6. The system for treating surfaces of bodies according to claim 1, characterized in that the system comprises a chamber which defines the space in which negative pressure can be generated.

7. The system for treating surfaces of bodies according to claim 1, characterized in that the system comprises a negative pressure generator, in particular a vacuum pump, for generating negative pressure in the space, wherein the negative pressure generator can be connected or is connected fluidically to the space by means of the connection means (5a).

8. The system for treating surfaces of bodies according to claim 1, characterized in that the device for plasma generation (8, 8a, 15) is designed to generate a dielectric barrier discharge.

9. The system for treating surfaces of bodies according to claim 1, characterized in that the device for plasma generation (8, 8a, 15) comprises at least one inner electrode (12, 21) and at least one earthed outer electrode (11).

10. The system for treating surfaces of bodies according to claim 9, characterized in that the outer electrode (11) comprises an electroconductive textile, in particular woven, material or consists of the latter.

11. The system for treating surfaces of bodies according to claim 1, wherein the outer electrode (11) comprises electroconductive gauze or consists of the latter.

12. The system for treating surfaces of bodies according to claim 1, characterized in that the device for plasma generation (8, 8a, 15) comprises a plurality of spacer elements (13) made from an electrically insulating material, wherein the spacer elements (13) define a distance between the inner electrode (12, 21) and the outer electrode (11).

13. The system for treating surfaces of bodies according to claim 1, characterized in that the system comprises a film (4); in particular a transparent film (4), for sealing and covering the system from the environment, wherein in particular the film (4) comprises at least one opening (4a) to which the connection means (5a) is connected, so that the negative pressure generator is in fluidic connection through the opening (4a) to the space in which negative pressure can be generated.

14. A method for treating surfaces of bodies, in particular for treating surfaces of human or animal tissue outside the human or animal body, wherein
i) a body with a surface to be treated is provided,
ii) a system for treating surfaces of bodies according to claim 1 is provided,
iii) a negative pressure is generated in a space positioned on the surface of the body,
v) the space is filled at least partly with plasma or plasma-activated medium.

15. The method for treating surfaces of bodies according to claim 14, wherein the space is filled alternately to the negative pressure generation with the plasma or the plasma-activated medium.
